(19) United States
(12) Patent Application Publication
(10) Pub. No.: US 2016/0317154 A1
(43) Pub. Date: Nov. 3, 2016

(54) ATRIAL-APPENDAGE LIGATION TREATMENT TOOL AND ATRIAL-APPENDAGE LIGATION SYSTEM

(71) Applicant: OLYMPUS CORPORATION, Tokyo (JP)

(72) Inventors: Takehiro KIMURA, Tokyo (JP); Kazutoshi KUMAGAI, Tokyo (JP); Yoshiro OKAZAKI, Tokyo (JP); Naoya SUGIMOTO, Tokyo (JP); Hiroki HIBINO, Tokyo (JP); Yoshiyuki KUMADA, Tokyo (JP); Hiroshi FUKUDA, Tokyo (JP)

(73) Assignee: OLYMPUS CORPORATION, Tokyo (JP)

(21) Appl. No.: 15/208,283

(22) Filed: Jul. 12, 2016

Related U.S. Application Data
(63) Continuation of application No. PCT/JP2015/053836, filed on Feb. 12, 2015.

(30) Foreign Application Priority Data
Mar. 31, 2014 (JP) 2014-074156

(51) Int. Cl. A61B 17/12; A61B 8/06; A61B 5/0402; A61B 5/026; A61B 5/02; A61B 5/103; A61B 17/128; A61B 8/12

(52) U.S. Cl. CPC A61B 17/12013; A61B 17/1285; A61B 8/065; A61B 8/12; A61B 5/0261; A61B 5/02007; A61B 5/1032; A61B 5/0402; A61B 2017/00044

(57) ABSTRACT

Completion of atrial-appendage ligation using a ligation loop is confirmed easily and more reliably. Provided is an atrial-appendage ligation treatment tool including: an insertion portion that is introduced into a cardiac sac through a sheath penetrated through a pericardium; a securing part that secures a distal end of the insertion portion to an atrial appendage; and a sensor that is provided at the distal end of the insertion portion and that detects the state of the atrial appendage.

### CROSS-REFERENCE TO RELATED APPLICATIONS

This is a continuation of International Application PCT/JP2015/053836 which is hereby incorporated by reference herein in its entirety.

This application is based on Japanese Patent Application No. 2014-074156, the contents of which are incorporated herein by reference.

### TECHNICAL FIELD

The present invention relates to an atrial-appendage ligation treatment tool and an atrial-appendage ligation system.

### BACKGROUND ART

In recent years, the number of patients with atrial fibrillation, which is one type of arrhythmia, has exhibited an increasing tendency. It is considered that brain infarction caused by atrial fibrillation occurs when a blood vessel in the brain is blocked by a blood clot formed in the heart (mainly, in the left atrial appendage). The most-frequent embolic source for the cardiogenic cerebral embolism is a blood clot in the left atrial appendage due to atrial fibrillation.

A common treatment recommended for prevention of cardiogenic cerebral embolisms is blood anticoagulant therapy with warfarin potassium; however, the administration of warfarin potassium is difficult to manage, and warfarin potassium has a risk of bleeding complications. As a substitute for this, a method of preventing embolisms by occluding the left atrial appendage has been developed (for example, Watchman, Boston Scientific). This is a jellyfish-shaped device for occluding the left atrial appendage, like a transvascular catheter.

On the other hand, a treatment tool for ligating the atrial appendage from outside the heart, without using an anticoagulant drug and without entering a blood vessel, is known (for example, see PTL 1). This is a treatment tool in which forceps and a ligation loop are inserted into the cardiac sac from outside the body, the ligation loop is looped around the atrial appendage while an end portion of the atrial appendage is being grasped and pulled by using the grasping forceps, and then the ligation loop is tightened, thus ligating the atrial appendage.

This treatment tool is provided with a sleeve that accommodates the ligation loop in a concave portion thereof, so as to facilitate looping of the ligation loop around the atrial appendage by keeping the ligation loop spread by using the sleeve.

### CITATION LIST

### Patent Literature

### {PTL 1} US Patent Application, Publication No. 2008/0294175

### SUMMARY OF INVENTION

### Solution to Problem

According to one aspect, the present invention provides an atrial-appendage ligation treatment tool including: an insertion portion that is introduced into a cardiac sac through a sheath penetrated through a pericardium; a securing part that secures a distal end of the insertion portion to an atrial appendage; and a sensor that is provided at the distal end of the insertion portion and that detects the state of the atrial appendage.

### BRIEF DESCRIPTION OF DRAWINGS

FIG. 1 is a perspective view showing an atrial-appendage ligation treatment tool according to one embodiment of the present invention.

FIG. 2 is a longitudinal sectional view showing a state where the atrial-appendage ligation treatment tool shown in FIG. 1 is accommodated in a sheath.

FIG. 3 is a front view showing an ultrasound-wave irradiation area produced by a sensor of the atrial-appendage ligation treatment tool shown in FIG. 1.

FIG. 4 is a view showing a state where a pressing portion of the atrial-appendage ligation treatment tool shown in FIG. 1 is brought close to the atrial appendage in the cardiac sac.

FIG. 5 is a view showing a state where the atrial appendage is sandwiched between two contact parts of the pressing portion of the atrial-appendage ligation treatment tool shown in FIG. 1.

FIG. 6 is a side view showing the ultrasound-wave irradiation area, with the atrial appendage being sandwiched between the two contact parts of the atrial-appendage ligation treatment tool shown in FIG. 1.

FIG. 7 is a view for explaining a state where a ligation loop is looped around the atrial appendage from the state in FIG. 5.

FIG. 8 is a side view for explaining detection of blood flow in the atrial appendage immediately after the ligation using the ligation loop looped around the atrial appendage in FIG. 7.

FIG. 9 is a side view for explaining detection of blood flow in the atrial appendage after the ligation using the ligation loop looped around the atrial appendage in FIG. 7.

FIG. 10A is a perspective view showing a rectangular-frame-shaped pressing portion according to a first modification of the pressing portion of the atrial-appendage ligation treatment tool shown in FIG. 1.

FIG. 10B is a perspective view showing an oval-frame-shaped pressing portion according to the first modification of the pressing portion of the atrial-appendage ligation treatment tool shown in FIG. 1.

FIG. 10C is a perspective view showing a C-shaped pressing portion according to the first modification of the pressing portion of the atrial-appendage ligation treatment tool shown in FIG. 1.

FIG. 10D is a perspective view showing a V-shaped pressing portion according to the first modification of the pressing portion of the atrial-appendage ligation treatment tool shown in FIG. 1.

FIG. 10E is a perspective view showing a pressing portion that has a shape in which only distal ends thereof are spread, according to the first modification of the pressing portion of the atrial-appendage ligation treatment tool shown in FIG. 1.

FIG. 11 is a perspective view showing a pressing portion in which the two contact parts are opened and closed, according to a second modification of the atrial-appendage ligation treatment tool shown in FIG. 1.

FIG. 12 is a front view showing a pressing portion in which the contact parts are made to attract each other by a magnet, according to a third modification of the atrial-appendage ligation treatment tool shown in FIG. 1.

FIG. 13A is a perspective view showing, in another method of bending a shaft, a state where a hollow shaft is rectified by a high-stiffness rod, according to a fourth modification of the atrial-appendage ligation treatment tool shown in FIG. 1.

FIG. 13B is a perspective view showing, in the method of bending the shaft, a state where the rod is withdrawn, thus letting the hollow shaft bend by means of a pre-bent section, according to the fourth modification of the atrial-appendage ligation treatment tool shown in FIG. 1.

FIG. 14 is a perspective view showing a pressing portion in which the pressing portion is bent by using a joint, according to a fifth modification of the atrial-appendage ligation treatment tool shown in FIG. 1.

FIG. 15 is a perspective view showing a pressing portion in which the bending direction is different from that of FIG. 14, according to a sixth modification of the atrial-appendage ligation treatment tool shown in FIG. 1.

FIG. 16 is a perspective view showing a pressing portion in which bending of the pressing portion with respect to the shaft and opening/closing of the two contact parts are performed by using joints, according to a seventh modification of the atrial-appendage ligation treatment tool shown in FIG. 1.

FIG. 17A is a perspective view showing a pressing portion in which bending of the pressing portion with respect to the shaft and opening/closing of the two contact parts are performed by using the joints, and a state where the pressing portion is linearly extended, according to an eighth modification of the atrial-appendage ligation treatment tool shown in FIG. 1.

FIG. 17B is a perspective view showing the pressing portion in which bending of the pressing portion with respect to the shaft and opening/closing of the two contact parts are performed by using the joints, and a state where only the contact parts are bent, according to the eighth modification of the atrial-appendage ligation treatment tool shown in FIG. 1.

FIG. 17C is a perspective view showing the pressing portion in which bending of the pressing portion with respect to the shaft and opening/closing of the two contact parts are performed by using the joints, and a state where only the contact parts are opened and closed, according to the eighth modification of the atrial-appendage ligation treatment tool shown in FIG. 1.

FIG. 18A is a perspective view showing the overall configuration of an atrial-appendage ligation treatment tool that integrally holds the ligation loop, according to a ninth modification of the atrial-appendage ligation treatment tool shown in FIG. 1.

FIG. 18B is an enlarged view of a holding part in the atrial-appendage ligation treatment tool that integrally holds the ligation loop, according to the ninth modification of the atrial-appendage ligation treatment tool shown in FIG. 1.

FIG. 19A is a perspective view showing the overall configuration of an atrial-appendage ligation treatment tool that integrally holds two ligation loops, according to a modification of the atrial-appendage ligation treatment tool shown in FIG. 18A.

FIG. 19B is an enlarged view of a holding part in the atrial-appendage ligation treatment tool that integrally holds the two ligation loops, according to the modification of the atrial-appendage ligation treatment tool shown in FIG. 18B.

FIG. 20 is a partial longitudinal sectional view showing an atrial-appendage ligation treatment tool in which the ligation loop forms a securing part, and an ultrasound sensor is provided on a side surface of the shaft, according to a tenth modification of the atrial-appendage ligation treatment tool shown in FIG. 1.

FIG. 21 is a partial longitudinal sectional view showing an atrial-appendage ligation treatment tool in which the ligation loop forms a securing part, and a prism is provided on a side surface of the shaft, according to an eleventh modification of the atrial-appendage ligation treatment tool shown in FIG. 1.

FIG. 22 is a cross-sectional view showing an example of the shaft shown in FIG. 20 or FIG. 21, according to a twelfth modification of the atrial-appendage ligation treatment tool shown in FIG. 1.

FIG. 23 is a partial perspective view showing an anti-rotation part of the shaft shown in FIG. 20 or FIG. 21, according to a thirteenth modification of the atrial-appendage ligation treatment tool shown in FIG. 1.

FIG. 24 is a partial longitudinal sectional view showing an atrial-appendage ligation treatment tool in which a balloon for pressing the sensor shown in FIG. 20 or FIG. 21 against the atrial appendage is provided, according to a fourteenth modification of the atrial-appendage ligation treatment tool shown in FIG. 1.

FIG. 25 is a side view showing a structure in which sensors are provided on grasping forceps, according to a fourteenth modification of the atrial-appendage ligation treatment tool shown in FIG. 1.

FIG. 26 is a block diagram showing an atrial-appendage ligation system according to the embodiment of the present invention.

### DESCRIPTION OF EMBODIMENT

An atrial-appendage ligation treatment tool 1 according to one embodiment of the present invention will be described below with reference to the drawings.

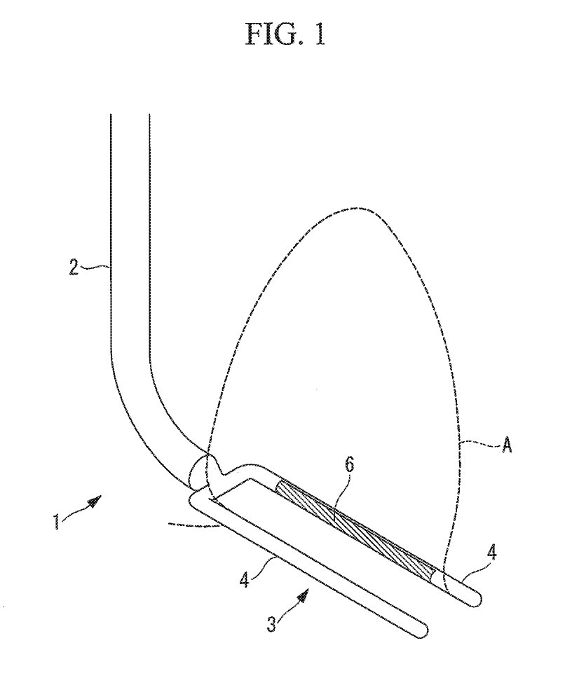

As shown in FIG. 1, the atrial-appendage ligation treatment tool 1 of this embodiment is provided with: an elongated shaft (insertion portion) 2 that is formed of an elastic material and that has a pre-bent section whose the distal end is bent at about 90 degrees; and a pressing portion 3 that is disposed at the distal end of the shaft 2. In this embodiment, the shaft 2 itself forms a biasing part for biasing in the direction in which it is bent due to the pre-bent section.

The pressing portion 3 is formed into a two-pronged shape having two straight-rod-like contact parts 4 that are disposed in parallel, with a predetermined space therebetween. A curvature plane of the shaft 2 produced by the pre-bent section is approximately perpendicular to a plane in which the two contact parts 4 are disposed.

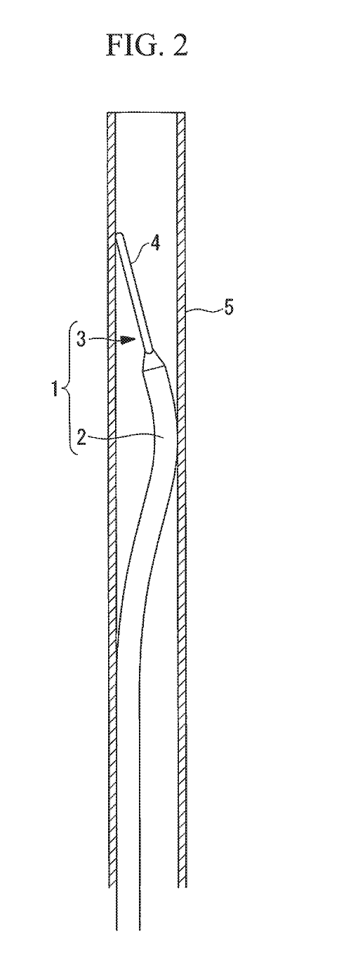

Although the shaft 2 can be bent along the shape of a sheath 5, the shaft 2 has stiffness so as to be able to transfer, in the longitudinal direction, a pressing force applied to a base end of the sheath 5. Furthermore, the pre-bent section of the shaft 2 is linearly extended by being inserted into the sheath 5, as shown in FIG. 2, so that the pressing portion 3 can be disposed almost at an extension of the shaft 2 in the longitudinal direction and can be approximately aligned with the shaft 2.

As shown in FIG. 1, the contact parts 4 are each formed to be circular in cross section, and the distal ends of the contact parts 4 are rounded so as not to stick in tissue even when pressed against the tissue.

The space between the contact parts 4 is set to such a dimension that an atrial appendage A can be sandwiched therebetween in the thickness direction.

The two contact parts 4 of the pressing portion 3 form a securing part that restricts the shaft 2 from moving in the thickness direction of the atrial appendage A by sandwiching the atrial appendage A between the contact parts 4 in the thickness direction and that restricts the shaft 2 from moving in the longitudinal direction of the atrial appendage A by pressing the two contact parts 4 against a boundary portion between the atrial appendage A and the atrium.

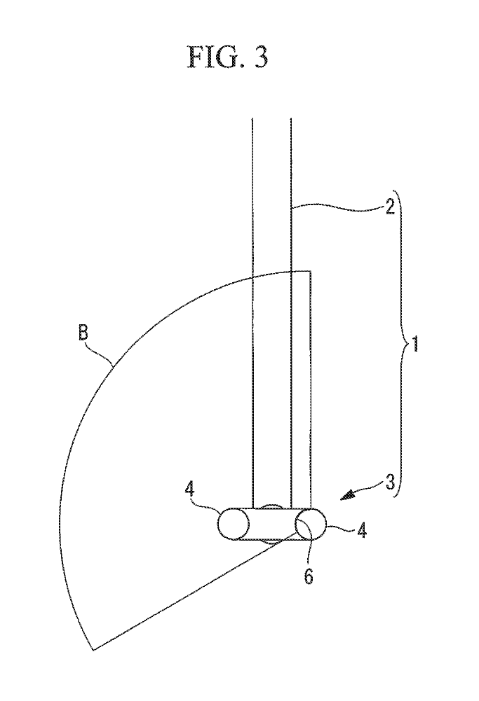

Furthermore, the atrial-appendage ligation treatment tool 1 of this embodiment is provided with a blood-flow sensor 6 formed of an ultrasound sensor that emits ultrasound waves and receives an echo, on a surface of one of the contact parts 4 that faces the other contact part 4. As shown in FIG. 3, the blood-flow sensor 6 has an ultrasound-wave irradiation area B that faces toward the other contact part 4 and that is biased toward the base end of the shaft 2.

The procedure for ligating the atrial appendage A by using the thus-configured atrial-appendage ligation treatment tool 1 of this embodiment will be described below.

In order to ligate the atrial appendage A by using the atrial-appendage ligation treatment tool 1 of this embodiment, first, in a state in which a distal-end opening of the sheath 5 is disposed in the cardiac sac after being made to penetrate through body-surface tissue and the pericardium from a lower portion of the ensiform cartilage, the atrial-appendage ligation treatment tool 1, with the pre-bent section of the shaft 2 being extended approximately linearly, is inserted into the sheath 5 and is made to advance to the inside of the cardiac sac.

At this time, the pressing portion 3 is approximately aligned with the shaft 2, thus allowing smooth advance in the sheath 5. Then, when the pressing portion 3 comes out from the distal-end opening of the sheath 5 to the inside of the cardiac sac, the pre-bent section of the shaft 2 that has been restrained is released, thus bending, as shown in FIG. 1, and the pressing portion 3 is pointed in a direction intersecting the longitudinal direction of the shaft 2.

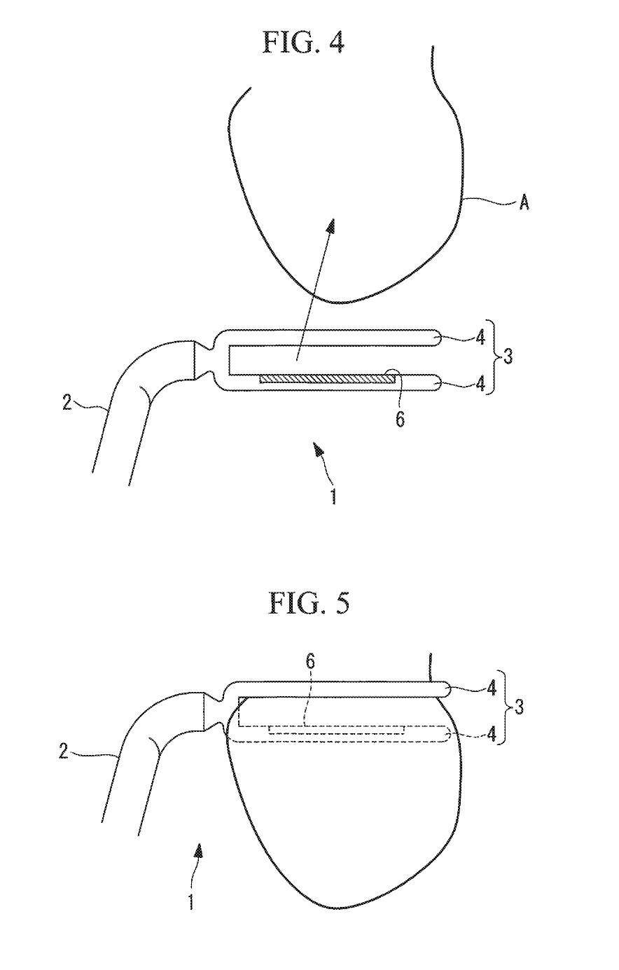

In this state, while performing observation with an endoscope (not shown) that is separately inserted into the cardiac sac, the shaft 2 is manipulated outside the body close to the base end of the sheath 5, the pressing portion 3 is brought close to the atrial appendage A from a distal end of the atrial appendage A, as shown in FIG. 4, and the atrial appendage A is inserted between the two contact parts 4 of the pressing portion 3, as shown in FIG. 5. Specifically, the shaft 2 is disposed at the right side of the left atrial appendage A (at the left side when viewed from the front). If observation with the endoscope is difficult, the shaft 2 may be moved to a location other than the right side of the left atrial appendage A, for example, to a location thereabove. Because the atrial appendage A is a sac-like tissue projecting, like an ear, from the outer surface of the heart and has flexibility, the atrial appendage A can be inserted between the contact parts 4 while being deformed when the pressing portion 3, which has stiffness, is pressed against the atrial appendage A. Then, the pressing portion 3 is made to advance while the distal end of the atrial appendage A is being pulled by the grasping forceps 7 introduced through the sheath 5, thereby making it possible to press the contact parts 4 of the pressing portion 3 against the outer wall of the left atrium and to stretch the atrial appendage A.

Then, as shown in FIG. 6, the contact parts 4 are disposed in the vicinity of the base of the atrial appendage A, and the atrial appendage A is sandwiched between the contact parts 4, thereby making it possible to stably press the contact parts 4, with the atrial appendage A being sandwiched therebetween, against the outer wall of the left atrium, even with heartbeats, and to press the surface of the atrial appendage A to make the atrial appendage A concave so as not to increase the dimension in the thickness direction.

In this case, the blood-flow sensor 6, which is provided on the contact part 4, is brought into close contact with the surface of the atrial appendage A made concave, and thus, as shown in FIG. 6, ultrasound waves can be reliably propagated to the atrial appendage A by arranging the ultrasound-wave irradiation area B toward the atrial appendage A side. In this state, ultrasound waves are radiated as needed, thereby making it possible to detect the blood flow in the atrial appendage A before ligation.

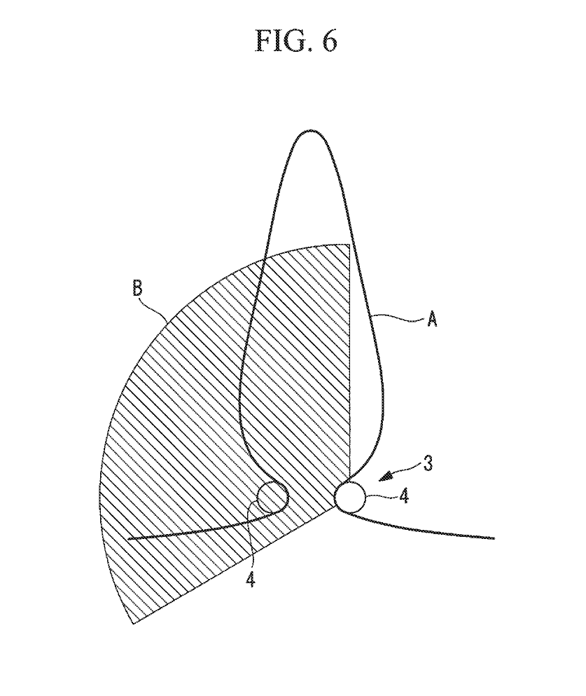

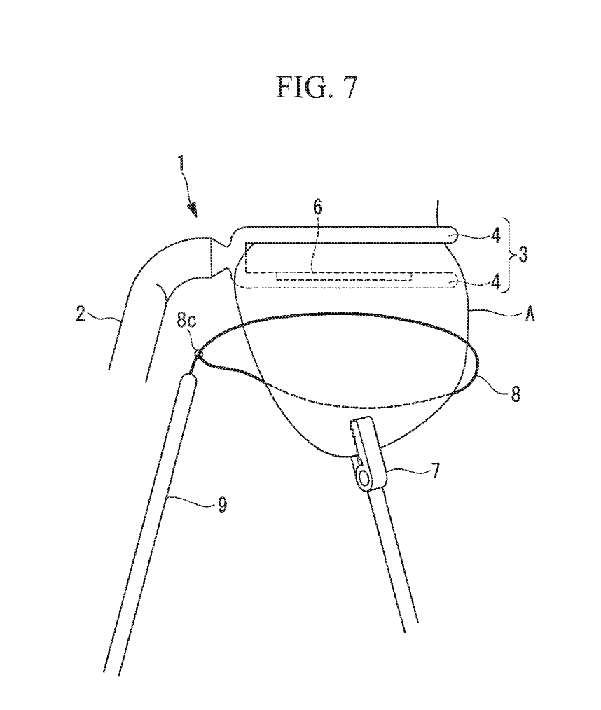

Then, in this state, as shown in FIG. 7, a shaft (hereinafter, referred to as loop shaft) 9 of a ligation loop 8 that is brought close to the atrial appendage A from an outer side of the grasping forceps 7 and the grasping forceps 7 are simultaneously manipulated to loop the ligation loop 8 around the atrial appendage A.

Specifically, with the end portion of the atrial appendage A being grasped by the grasping forceps 7, the loop shaft 9 is pushed at the same time as the grasping forceps 7 is pulled, and the grasping forceps 7 grasping the atrial appendage A is moved so as to be pulled out from the inside of the ligation loop 8, thereby making it possible to insert the atrial appendage A into the ligation loop 8 and to easily loop the ligation loop 8 around the atrial appendage A, as shown in FIG. 7.

When the ligation loop 8 is positioned at the base portion of the atrial appendage A, which is stretched and exposed by being pulled by the grasping forceps 7 while the pressing portion 3 is being pressed against the outer wall of the left atrium, one end of the ligation loop 8 is pulled out from the base end of the loop shaft 9 while the loop shaft 9 is being pushed, thereby tightening the ligation loop 8 and ligating the atrial appendage A.

In this case, the pressing portion 3 of the atrial-appendage ligation treatment tool 1 is disposed in the vicinity of the position where the atrial appendage A is to be ligated, thus reducing the dimension of the atrial appendage A in the thickness direction; therefore, it is possible to hold the ligation position such that the ligation loop 8 does not come off toward the distal end of the atrial appendage A due to the tightening thereof.

If the distal end of the atrial appendage A is pulled by the grasping forceps 7 without using the atrial-appendage ligation treatment tool 1, the outer wall of the left atrium is also pulled together with the atrial appendage A; therefore, the atrial appendage A is not sufficiently stretched, and the base portion of the atrial appendage A, which is to be ligated, is not exposed. On the other hand, according to this embodiment, as shown in FIG. 7, the pressing portion 3 sandwiches the vicinity of the base of the atrial appendage A in the thickness direction and is pressed against the outer wall of the left atrium; therefore, the atrial appendage A can be sufficiently stretched by being pulled by the grasping forceps 7, and the base portion thereof can be exposed. Furthermore, because the surface of the atrial appendage A is made concave by the pressing portion 3, the ligation loop 8 can remain at this position without being slipped toward the distal end. Accordingly, there is an advantage that the atrial appendage A can be ligated at a position as close to the base thereof as possible.

As shown in FIG. 8, after the atrial appendage A is ligated, the contact parts 4 are disposed closer to the base end of the atrial appendage A than the ligation loop 8 is, and the blood-flow sensor 6 is activated. Accordingly, it is possible to arrange the ultrasound-wave irradiation area B toward an interior region of the atrial appendage A that is closer to the distal end thereof than the ligation loop 8 is, through an interior region of the atrial appendage A that is closer to the base end thereof than the ligation loop 8 is, and to more reliably detect the blood flow in the ligated atrial appendage A.

In this case, after the atrial appendage A is ligated, as shown in FIG. 9, the pressing portion 3 is slightly pulled back toward the distal end of the atrial appendage A, thereby disposing the contact parts 4 at a position closer to the distal end of the atrial appendage A than the position on the atrial appendage A ligated by the ligation loop 8 is, and the blood-flow sensor 6 is activated. Accordingly, the ultrasound-wave irradiation area B can be directly arranged toward the region of the atrial appendage A that is closer to the distal end thereof than the ligation loop 8 is. Thus, it is possible to prevent a situation in which ultrasound waves do not propagate to the interior region of the atrial appendage A that is closer to the distal end thereof than the ligation loop 8 is, through the interior region of the atrial appendage A that is closer to the base end thereof than the ligation loop 8 is, and to more reliably detect the blood flow in the ligated atrial appendage A.

In this case, when the existence of the blood flow in the atrial appendage A is detected by the blood-flow sensor 6, the ligation loop 8 can be further tightened to further ensure the ligation. Furthermore, when it is confirmed that the blood flow does not exist, the ligation loop 8 does not need to be tightened any further, thus making it possible to prevent damage to the atrial-appendage tissue at the ligated portion, which would be caused by excessive tightening.

After the ligation is confirmed, the loop shaft 9 is withdrawn, with the ligation loop 8 remaining, and the ligation loop 8 is cut in the vicinity of a knot 8c by using scissors forceps (not shown) introduced through the sheath 5.

After that, by moving the shaft 2 of the atrial-appendage ligation treatment tool 1 in a direction in which it is withdrawn from the sheath 5, the pressing portion 3 is removed from the atrial appendage A, is drawn into the sheath 5 while the pre-bent section is being rectified, and is taken out to the outside of the body through the sheath 5. Then, all treatment tools are withdrawn, thereby finishing the treatment.

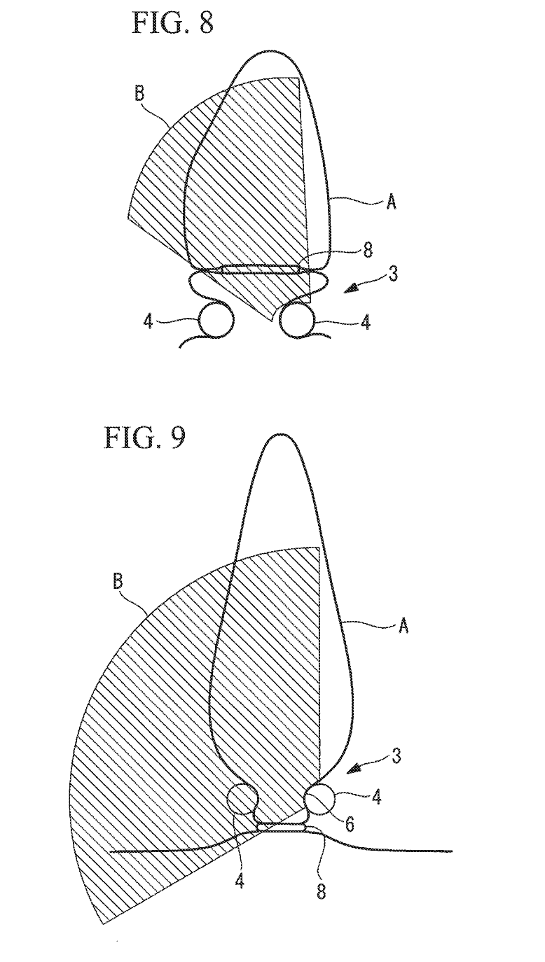

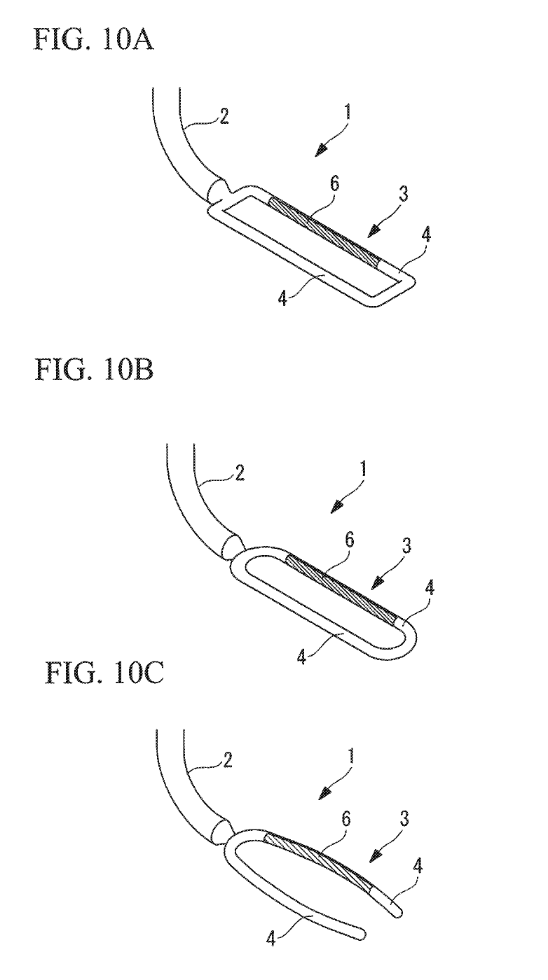

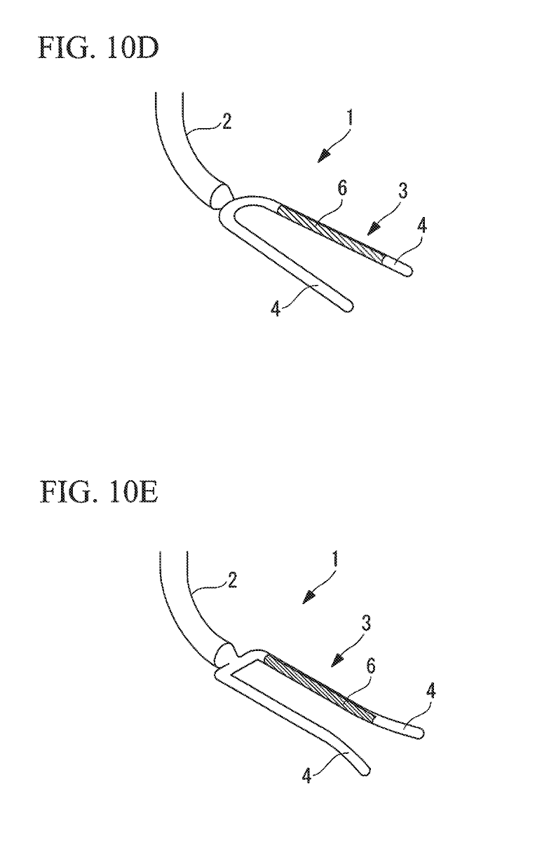

Note that, in this embodiment, the pressing portion 3, which has the two straight-rod-like contact parts 4 disposed in parallel, with a space therebetween, is shown as an example; however, the present invention is not limited thereto, as shown in FIGS. 10A to 10E, it is possible to adopt a pressing portion having a desired form, e.g., a closed rectangular frame-shaped form in which the distal ends of the two straight-rod-like contact parts 4 are connected, shown in FIG. 10A; an oval form shown in FIG. 10B; a substantially C-shaped form with two curved rod-like contact parts 4 shown in FIG. 10C; a substantially V-shaped form in which the space between the two straight-rod-like contact parts 4 increases toward the distal ends thereof, shown in FIG. 10D; and a form in which the distal ends of the two straight-rod-like contact parts 4 are spread outward, as shown in FIG. 10E.

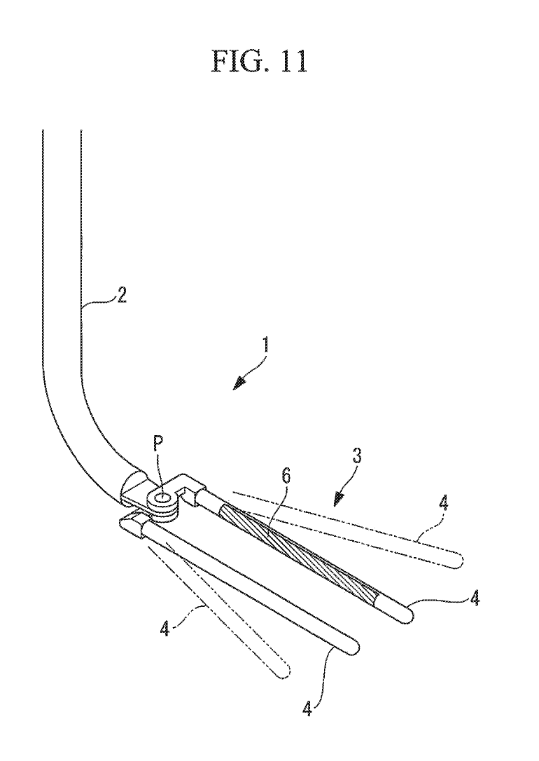

Furthermore, a description has been given of the pressing portion 3 in which the two contact parts 4 are fixed at the positions with the predetermined space therebetween; however, instead of this, as shown in FIG. 11, it is possible to provide a pressing portion 3 in which the two contact parts 4 are each rotated about the shaft of a joint P, thereby allowing the space between the contact parts 4 to be changed (the contact parts 4 to be opened/closed).

By doing so, it is possible to bring the two contact parts 4 close to each other to make them compact when passing through the sheath 5; to widen the space between the two contact parts 4 when the atrial appendage A is to be inserted therebetween, thus facilitating the insertion; to again bring the two contact parts 4 close to each other after the insertion, thus sandwiching the atrial appendage A in the thickness direction; to press the outer wall of the left atrium while the atrial appendage A is being tied up; and to bring the blood-flow sensor 6 into close contact with the surface of the atrial appendage A.

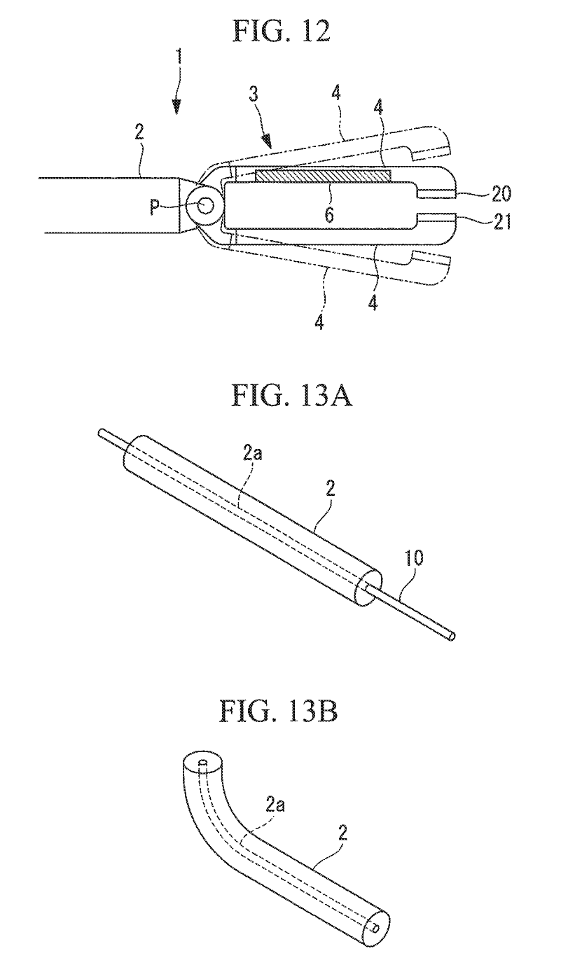

As a mechanism for opening and closing the two contact parts 4, it is possible to adopt a mechanism for opening and closing them by using a wire (not shown) that is introduced along the shaft 2, as in normal grasping forceps, or a mechanism in which a distal-end portion 20 of one of the two contact parts 4 is formed of a magnetic material and the other can be magnetized by an electromagnet 21, as shown in FIG. 12. After the atrial appendage A is inserted therebetween while the electromagnet 21 has not yet been magnetized, the contact part 4 is magnetized by the electromagnet 21, thus bringing the two contact parts 4 close to each other due to the magnetic force, the atrial appendage A is firmly sandwiched in the thickness direction to be tied up, and thus the blood-flow sensor 6 can be brought into close contact with the atrial appendage A.

Note that the electromagnet 21 may be provided on both of the two contact parts 4. Furthermore, in a case in which the two contact parts 4 are introduced through separate shafts 2, permanent magnets can be adopted instead of the electromagnets 21.

Furthermore, in this embodiment, although the pre-bent section of the shaft 2 is rectified by using the sheath 5, instead of this, as shown in FIGS. 13A and 13B, it is also possible to removably insert a straight rod 10 that has higher stiffness than the shaft 2 into a through-hole 2a penetrating the shaft 2 in the longitudinal direction thereof, thus rectifying the shaft 2, and to withdraw the rod 10 from the through-hole 2a, thereby letting the shaft 2 bend due to the pre-bent section. Accordingly, compared with the case in which the shaft 2 is rectified by using the sheath 5, the friction force with respect to the sheath 5 is reduced, thus making it possible to improve the ease of introducing and withdrawing the shaft 2 to and from the cardiac sac.

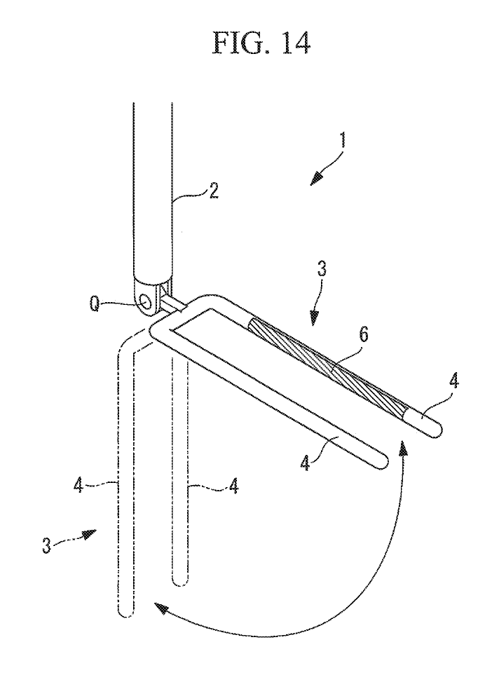

Furthermore, in this embodiment, the shaft 2, which is formed of an elastic material and has the pre-bent section, allows the pressing portion 3 to be disposed in the cardiac sac in a direction intersecting the longitudinal direction of the shaft 2. Instead of this, as shown in FIG. 14, it is possible to provide a joint Q between the shaft 2 and the pressing portion 3 and to bias the joint Q so that the joint Q is rotated by using a biasing part (not shown), such as a spring. At this time, the direction of rotation of the joint Q is along a plane perpendicular to a plane that includes the two contact parts 4.

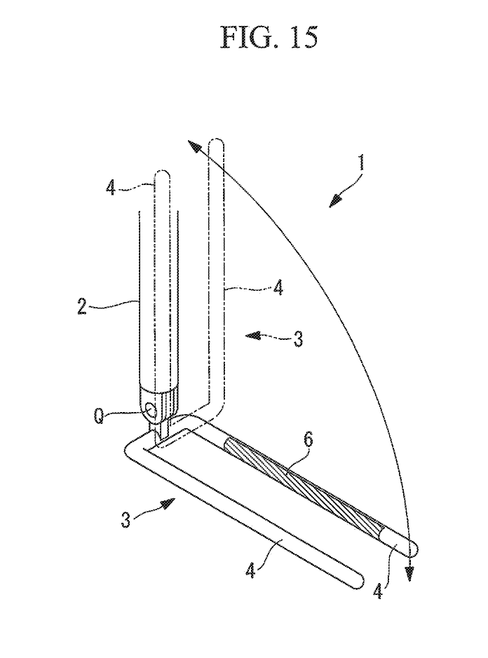

As indicated by a chain line in FIG. 14, the joint Q may be rotated between a state where the pressing portion is extended to be in a line on an extension of the shaft 2 and a state where the pressing portion 3 is disposed at a position to be substantially perpendicular to the longitudinal direction of the shaft 2. Alternatively, as shown in FIG. 15, the joint Q may be rotated between a state where the pressing portion 3 is folded toward the shaft 2 and a state where the pressing portion 3 is disposed at a position to be substantially perpendicular to the longitudinal direction of the shaft 2. In the case of FIG. 15, it is preferable that a wire (not shown) used for returning to the folded state be provided.

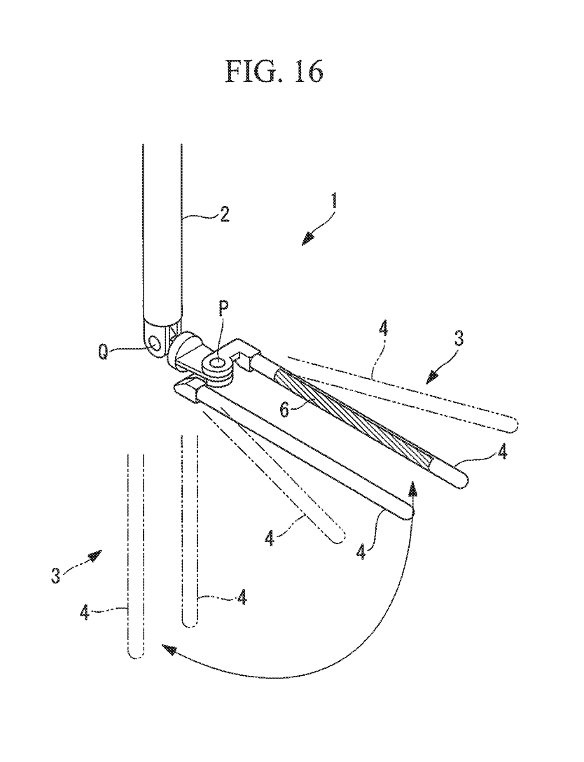

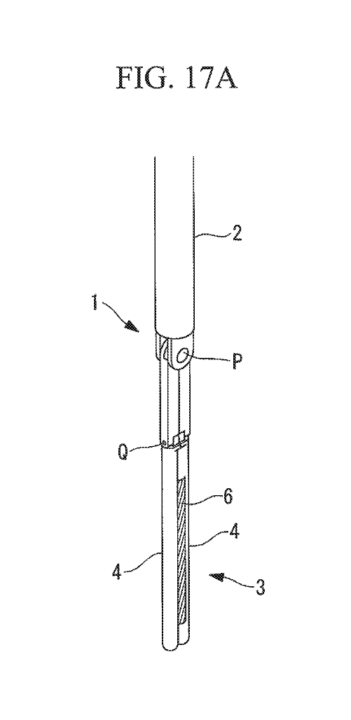

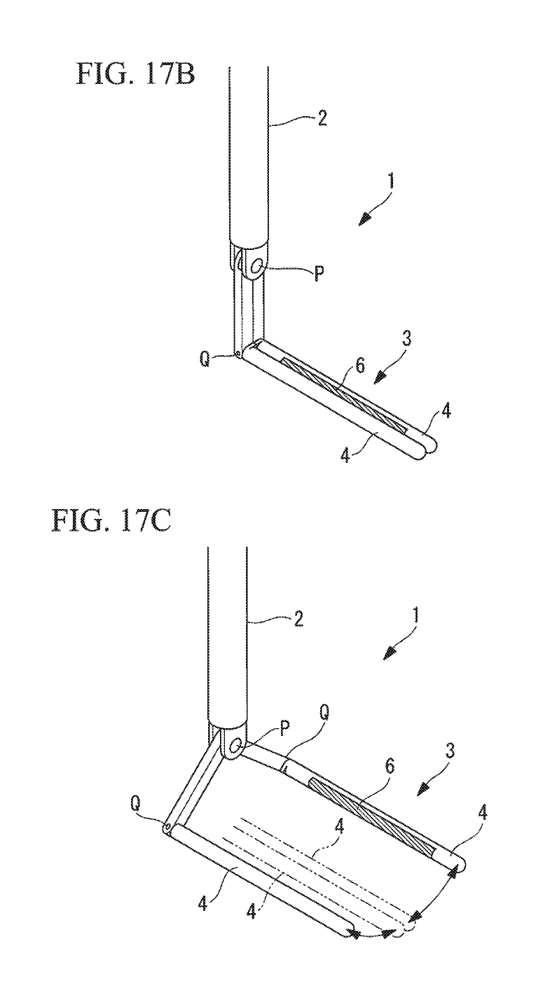

In order to change the angle formed by the shaft 2 and the pressing portion 3 and to allow the pressing portion 3 to be opened and closed by using the joints P and Q, as shown in FIG. 16, it is possible to provide the joint Q, which allows the entire pressing portion 3 to rotate with respect to the shaft 2, at a position that is closer to the base end than the joint P, which allows the two contact parts 4 of the pressing portion 3 to be opened and closed, is; alternatively, as shown in FIGS. 17A to 17C, it is possible to provide the joint P, which allows the two contact parts 4 to be opened and closed, at a position that is closer to the base end than the joint Q, which allows the contact parts 4 to rotate with respect to the shaft 2, is.

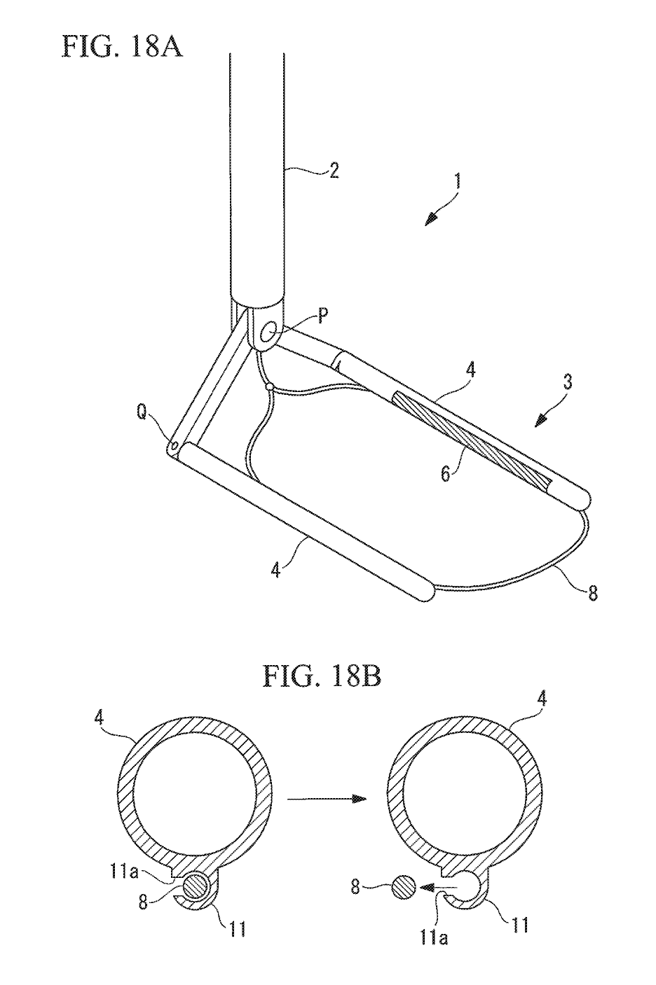

Furthermore, in this embodiment, a description has been given of an example case in which the atrial-appendage ligation treatment tool 1 is separated from the ligation loop 8; however, instead of this, it is possible to adopt an atrial-appendage ligation treatment tool 1 that is integrally provided with the ligation loop 8, as shown in FIG. 18A.

In the example shown in FIG. 18A, the shaft 2 of the atrial-appendage ligation treatment tool 1 is formed to be hollow, thus serving as the loop shaft 9, and the contact parts 4 are provided with holding parts 11 that each hold part of the ligation loop 8.

As shown in FIG. 18B, the holding parts 11 each have a C-shaped cross-section so as to allow the ligation loop 8 to pass therethrough, and hold the ligation loop 8 therein via slits 11a that each have a width dimension slightly less than the outer-diameter dimension of the ligation loop 8. The holding parts 11 are disposed closer to the atrium than the contact parts 4 are, when the contact parts 4 are disposed along the width direction of the atrial appendage A.

Accordingly, before the ligation loop 8 has been tightened, the holding parts 11 hold the ligation loop 8 in the holding parts 11, and the space between the contact parts 4 is widened to spread the ligation loop 8, thus making it possible to facilitate insertion of the atrial appendage A thereinto. Furthermore, when the ligation loop 8 is tightened to ligate the atrial appendage A, the ligation loop 8 passes through the slits 11a of the holding parts 11 due to the tension thereof, thus being released from the held state produced by the holding parts 11.

Then, because the holding parts 11 are disposed closer to the atrium than the contact parts 4 are, the ligation loop 8 released from the holding parts 11 can ligate a portion of the atrial appendage A closer to the atrium than the contact parts 4 are. Therefore, it is possible to dispose the contact parts 4 and the blood-flow sensor 6, which is provided on the contact part 4, closer to the distal end of the atrial appendage A than the ligated portion, which is ligated by the ligation loop 8, is, and to detect the blood flow in the atrial appendage A without being obstructed by the ligation loop 8.

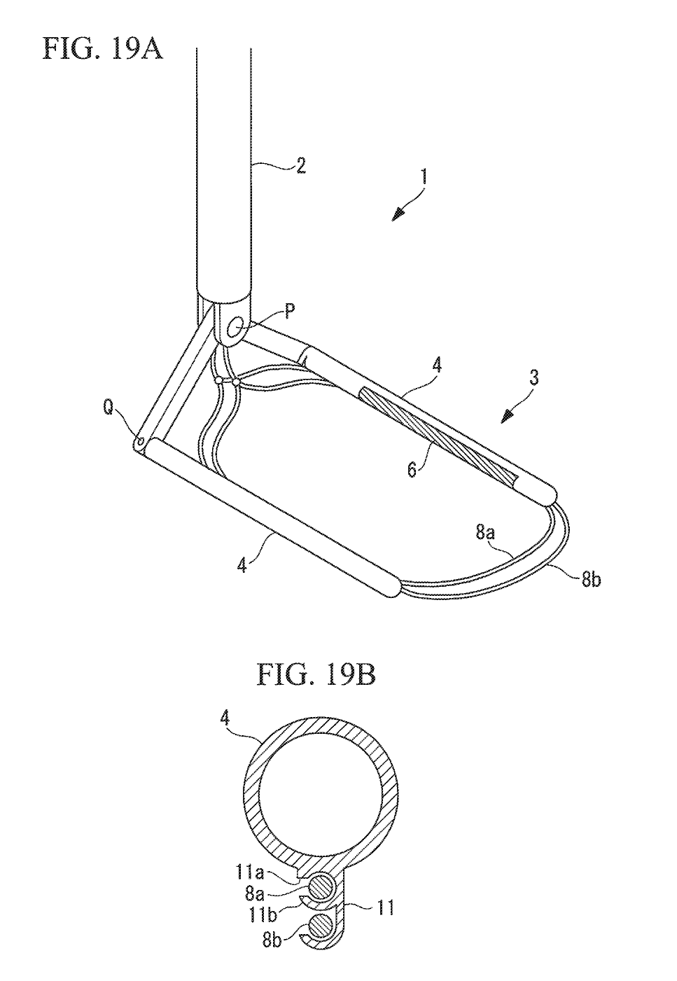

Furthermore, as shown in FIGS. 19A and 19B, two separate ligation loops 8a and 8b may be respectively accommodated in two slits 11a and 11b that are provided in each holding part. Since the inner-cavity surface of the atrial appendage has a complicated concave-convex shape, sufficient ligation cannot be performed with a single ligation loop in some cases. In those cases, according to this embodiment, ligation can be performed by using the two ligation loops, and it is not necessary to insert a new device into the cardiac sac in order to prepare another loop.

Furthermore, the ligation loops 8a and 8b may be of different types from each other. For example, when bioabsobable materials are used, two types of ligatures that have different absorption rates may be used. Alternatively, the ligatures may have different ligature thicknesses or numbers of strands, such as a single wire or a stranded wire. In this case, there is an advantageous effect in that a loop to be used for ligation can be selected after the state of the left atrial appendage is observed by using an endoscope etc.

Furthermore, in this embodiment, although the blood-flow sensor 6 is provided on one of the two contact parts 4, instead of this, it may be provided on both of them. Furthermore, in that case, one of the blood-flow sensors 6 may be a wave transmitting unit, and the other may be a wave receiving unit.

Furthermore, as the blood-flow sensor 6, it is possible to adopt an ultrasound sensor that utilizes the ultrasonic Doppler effect or an optical sensor that utilizes the optical Doppler effect. With the ultrasound sensor, there are advantages in that the degree of propagation is high, and the sensor is not influenced by the color of tissue. On the other hand, with the optical sensor, it is not necessary to firmly bring the blood-flow sensor 6 into close contact with the surface of the atrial appendage A, unlike the ultrasound sensor, thus making it possible to more easily detect the blood flow. Furthermore, in the case of the optical sensor, there are advantages in that the resolution can be improved by narrowing the light beam, there is no need to worry about electrical insulation at the interface with the tissue, and the sensor is less affected by the acoustic impedance (hardness) of the tissue.

Furthermore, in this embodiment, although the blood-flow sensor 6, which detects the blood flow, is used as an example sensor, instead of this, it is possible to adopt a sensor that measures the state of the atrial appendage A other than the blood flow, for example, the hardness of the atrial appendage A or the color of the atrial appendage A.

For hardness measurement, for example, a sensor using ultrasound elastography or shear wave imaging can be adopted. Furthermore, color measurement can be performed by using an electroscopic sensor or a colorimeter.

When the atrial appendage A is ligated, the tissue is hardened due to congestion; thus, the ligation state can be detected by measuring the hardness. Furthermore, when the atrial appendage A is ligated, the color changes due to congestion; thus, the ligation state can be detected by measuring the color.

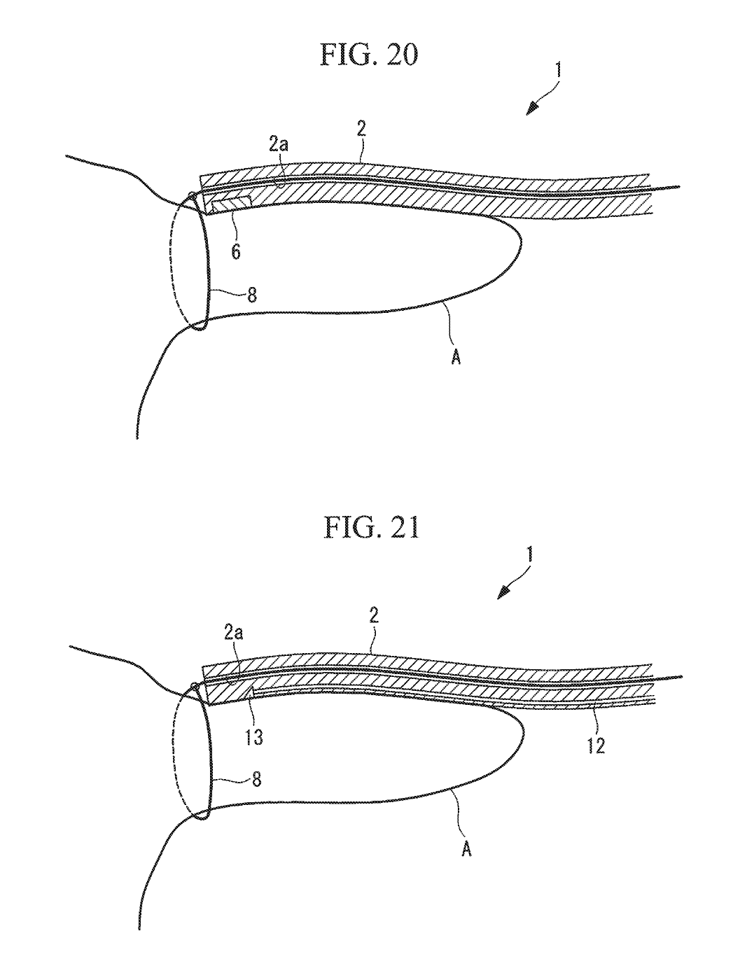

Furthermore, although the pressing portion 3, which has the two contact parts 4 to be disposed so as to sandwich the atrial appendage A, is shown as an example securing part, instead of this, as shown in FIG. 20, the ligation loop 8 may be used as the securing part. In that case, the blood-flow sensor 6 may be disposed on a side surface of the shaft 2.

The atrial appendage A is tightened by the ligation loop 8 that is introduced through the through-hole 2a provided in the shaft 2, thereby making it possible to secure the shaft 2 to the atrial appendage A and to stably detect the blood flow by using the blood-flow sensor 6.

By doing so, because the shaft 2 is secured to the atrial appendage A by tightening the ligation loop 8, detection of the blood flow is performed by the blood-flow sensor 6 to confirm the presence or absence of the blood flow, and, when the blood flow exists in the atrial appendage A, the ligation loop 8 is further tightened, thereby making it possible to perform more reliable ligation. Furthermore, as a result of the detection performed by the blood-flow sensor 6, when the blood flow does not exist in the atrial appendage A, excessive tightening of the ligation loop 8 is prevented, thus making it possible to maintain the health of the atrial appendage A.

In this case, because the blood-flow sensor 6 that is provided on the side surface of the shaft 2 is disposed closer to the base end of the shaft 2, i.e., closer to the distal end of the atrial appendage A, than the ligation loop 8 is, it is possible to arrange the ultrasound-wave irradiation area B produced by the blood-flow sensor 6 closer to the distal end of the atrial appendage A than the ligation loop 8 is, thus more reliably detecting the presence or absence of the blood flow in the atrial appendage A.

Furthermore, when an optical sensor is used as the blood-flow sensor 6, as shown in FIG. 21, it is necessary to dispose a prism 13 that polarizes light introduced through an optical fiber 12 provided on the shaft 2, to an outer side in a radial direction of the shaft 2.

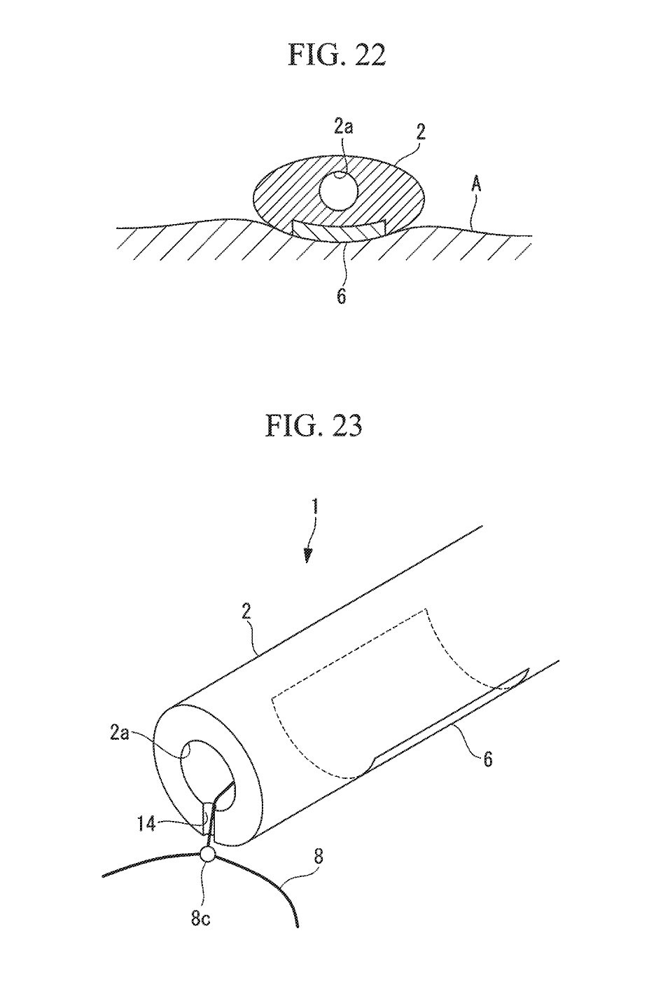

Furthermore, in order to stably dispose the blood-flow sensor 6 that is disposed on the side surface of the shaft 2, close to the surface of the atrial appendage A, as shown in FIG. 22, it is possible to form the shaft 2 into a flattened shape in cross-section and to dispose the blood-flow sensor 6 on a more-flattened portion. Note that, although an oval shape is shown in FIG. 22 as the flattened shape, the shape is not limited thereto, and a desired shape, such as a rectangle shape or an oval shape, can be adopted.

Similarly, when the atrial appendage A is ligated, the ligation loop 8 extends in any of the radial directions of the shaft 2, and the atrial appendage A is disposed in this direction, as shown in FIG. 23; thus, by using this arrangement, it is possible to provide a groove 14 that extends in one direction from the distal-end opening of the through-hole 2a of the shaft 2 to the side surface of the shaft 2 and to dispose the blood-flow sensor 6 on the side surface to which the groove 14 is connected. The groove 14 may have a size for allowing the wire which forms the ligation loop 8 to be accommodated therein.

The ligation loop 8 projecting forward from the distal-end opening of the shaft 2 is looped around the atrial appendage A, and a wire portion that is closer to the base end than the knot 8c of the ligation loop 8 is is accommodated in the groove 14 provided at the distal end of the shaft 2, thereby engaging the wire portion with the groove 14 and prohibiting the shaft 2 from rotating about the axis thereof. Because the blood-flow sensor 6 is provided on the side surface to which the groove 14 extends, the blood-flow sensor 6 is secured in the direction toward the surface of the atrial appendage A. Accordingly, the blood-flow sensor 6 on the side surface of the shaft 2 can be stably disposed close to the surface of the atrial appendage A.

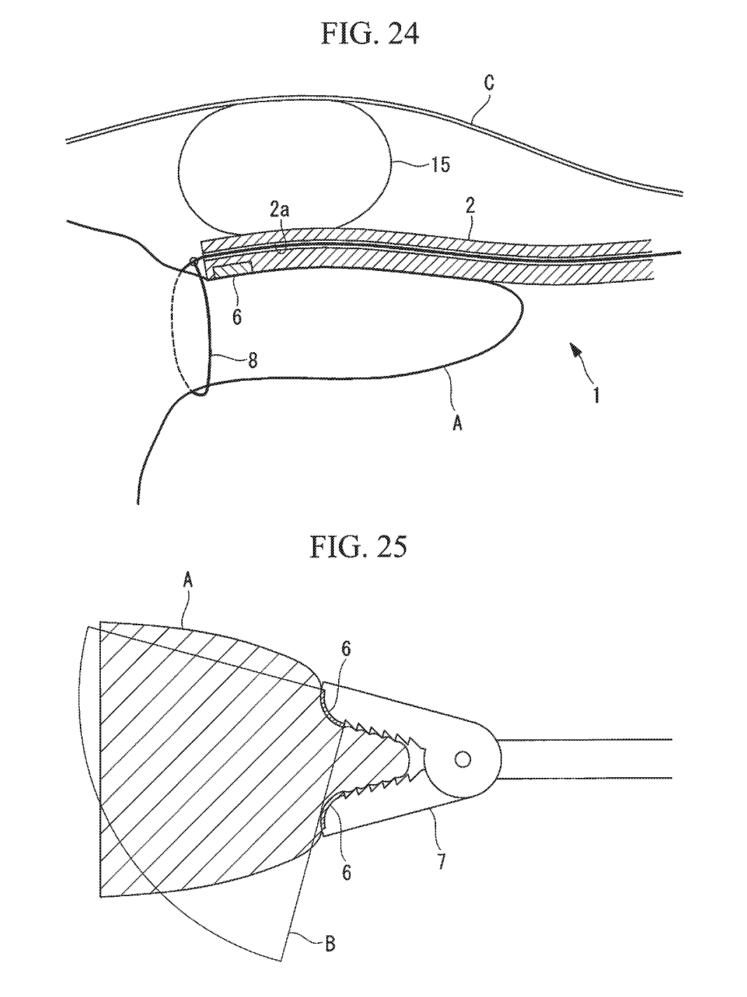

Furthermore, in order to more-reliably bring the blood-flow sensor 6, which is provided on the side surface of the shaft 2, into close contact with the surface of the atrial appendage A, as shown in FIG. 24, it is possible to provide an expandable balloon 15 on an opposite side surface of the shaft 2 from the blood-flow sensor 6. For example, the balloon 15 is expanded between the pericardium C and the shaft 2, thereby making it possible to press the shaft 2 against the atrial appendage A and to maintain a reliable contact state of the blood-flow sensor 6, for example.

Furthermore, in this embodiment, as shown in FIG. 25, the blood-flow sensor 6 may be disposed on grasping surfaces of the grasping forceps 7, which can grasp the atrial appendage A in the thickness direction. Because the grasping forceps 7 is used to grasp and pull the distal end of the atrial appendage A before the ligation loop 8 is looped around the atrial appendage A, the grasping forceps 7 may be attached to the shaft 2 of the ligation loop 8, which is disposed substantially parallel thereto, so as to be relatively movable in the longitudinal direction.

In this case, because the distal end of the atrial appendage A is grasped by the grasping forceps 7, it is preferable that the blood-flow sensor 6 have the irradiation area B that is arranged in the vicinity of the distal end of the grasping forceps 7 and that spreads forward. Furthermore, in order to be able to detect the blood flow in the atrial appendage A even when the distal end of the atrial appendage A is pulled in any direction when it is grasped and pulled, the blood-flow sensor 6 may be provided on both of the grasping surfaces of the grasping forceps 7.

Note that, in order to prevent detection performed by the blood-flow sensor 6 from being disturbed, it is preferable that the wire which forms the ligation loop 8 be formed of an ultrasound-propagating medium in the case where the blood-flow sensor 6 is an ultrasound sensor and be formed of an optically-transmissive wire in the case where the blood-flow sensor 6 is an optical sensor.

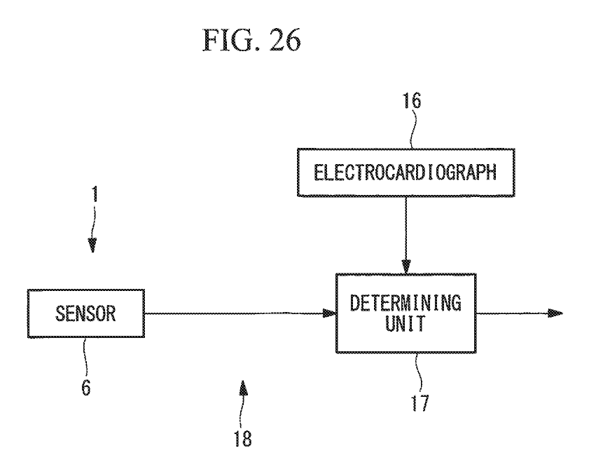

Furthermore, in this embodiment, as shown in FIG. 26, it is possible to configure an atrial-appendage ligation system 18 that is obtained by combining any of the above-described atrial-appendage ligation treatment tools 1, an electrocardiograph 16, and a determining unit 17.

The determining unit 17 compares an electrocardiographic signal output from the electrocardiograph 16 with the blood flow in the atrial appendage A detected by the blood-flow sensor 6 of the atrial-appendage ligation treatment tool 1, at the respective timings thereof, and determines whether the blood flow exists during the systole of the heart. As a result of the comparison, when the blood flow does not exist during the systole, it is possible to confirm that ligation has been effectively performed, and, when the blood flow exists at a timing other than the systole, it is possible to determine that the blood flow is noise.

Furthermore, the determining unit 17 may compare a heart rate in an electrocardiographic signal output from the electrocardiograph 16 with a detected rate detected by the blood-flow sensor 6. As a result of the comparison, if those rates do not match, it is possible to determine that either of the detected rates includes noise.

The above-described embodiment leads to the following inventions.

According to one aspect, the present invention provides an atrial-appendage ligation treatment tool including: an insertion portion that is introduced into a cardiac sac through a sheath penetrated through a pericardium; a securing part that secures a distal end of the insertion portion to an atrial appendage; and a sensor that is provided at the distal end of the insertion portion and that detects the state of the atrial appendage.

According to this aspect, the insertion portion of the atrial-appendage ligation treatment tool is inserted into the cardiac sac through the sheath penetrated through the pericardium, and, with the distal end of the insertion portion being secured to the atrial appendage by the securing part, the state of the atrial appendage can be detected by using the sensor, which is provided at the distal end of the insertion portion. For example, after ligation of the atrial appendage using a ligation loop, the state of the atrial appendage is detected, thereby making it possible to confirm completion of the ligation. In this case, because the distal end of the insertion portion, at which the sensor is provided, is secured to the atrial appendage by the securing part, the state of the atrial appendage can be easily and more reliably confirmed irrespective of beating of the atrial appendage.

In the above-described aspect, the sensor may be a blood-flow sensor that detects a blood flow in the atrial appendage.

By doing so, it is possible to detect a blood flow in the atrial appendage by using the blood-flow sensor secured to the atrial appendage and to determine that the ligation is insufficient when a blood flow exists in the ligated atrial appendage.

Furthermore, in the above-described aspect, the blood-flow sensor may be an ultrasound sensor.

By doing so, the ultrasound sensor is secured so as to be in close contact with the surface of the atrial appendage, to detect reflected waves or transmitted waves of ultrasound waves that have been radiated onto the inside of the atrial appendage, thereby making it possible to easily detect the blood flow in the atrial appendage due to the ultrasonic Doppler effect.

Furthermore, in the above-described aspect, the blood-flow sensor may be an optical sensor.

By doing so, the optical sensor is secured so as to face the surface of the atrial appendage, to detect reflected light or transmitted light of light that has been radiated onto an inside of the atrial appendage, thereby making it possible to easily detect the blood flow in the atrial appendage due to the optical Doppler effect. Furthermore, according to the optical sensor, it is possible to detect the blood flow irrespective of a contact state with the surface of the atrial appendage.

Furthermore, in the above-described aspect, the sensor may be a hardness sensor that measures the hardness of atrial-appendage tissue.

By doing so, by using the characteristic of atrial-appendage tissue to become hardened after ligation, the hardness of the atrial-appendage tissue before and after ligation is measured by the hardness sensor, thereby making it possible to easily detect completion of the ligation.

Furthermore, in the above-described aspect, the sensor may be a color-measuring sensor that measures the color of atrial-appendage tissue.

By doing so, by using the characteristic of atrial-appendage tissue to change color due to congestion after ligation, a change in color of the atrial-appendage tissue before and after ligation is measured by the color-measuring sensor, thereby making it possible to easily detect completion of the ligation.

Furthermore, in the above-described aspect, the securing part may include two contact parts that can be disposed at a position where the atrial appendage is to be sandwiched therebetween in the thickness direction.

By doing so, the two contact parts are disposed at a position where the atrial appendage is to be sandwiched therebetween in the thickness direction, thereby making it possible to secure the insertion portion so as not to be moved in the thickness direction of the atrial appendage. Furthermore, the contact parts are disposed in the vicinity of the boundary between the atrial appendage and the atrium and are pressed against the outer surface of the atrium, thereby making it possible to secure the insertion portion also in the longitudinal direction of the atrial appendage. Accordingly, it is possible to stably secure the sensor to the atrial appendage and to accurately detect the state of the atrial appendage.

Furthermore, in the above-described aspect, the contact parts may be provided such that the space therebetween can be changed.

By doing so, the space between the two contact parts is widened, thereby making it possible to easily dispose the contact parts at a position where the atrial appendage is to be sandwiched in the thickness direction; and, with the two contact parts being disposed at the position where the atrial appendage is to be sandwiched in the thickness direction, the space therebetween is narrowed, thereby making it possible to sandwich the atrial appendage in the thickness direction between the contact parts and to more reliably secure the distal end of the insertion portion to the atrial appendage.

Furthermore, in the above-described aspect, the sensor may be disposed at at least one of positions of the two contact parts that face each other.

By doing so, it is possible to secure the sensor to the atrial appendage so as to face toward the surface of the atrial appendage and to accurately detect the state of the atrial appendage by using the sensor.

Furthermore, in the above-described aspect, the sensor may be disposed at at least one of distal ends of the two contact parts.

By doing so, when the two contact parts are disposed at a position where the atrial appendage is to be sandwiched in the thickness direction, and the space therebetween is narrowed to sandwich the atrial appendage in the thickness direction, the distal ends of the contact parts dig into the outer surface of the atrial appendage, thereby making it possible to make the sensor, which is provided at the distal end, face toward the inside of the atrial appendage.

Furthermore, in the above-described aspect, the securing part may be a ligation loop that is introduced to the distal end of the insertion portion through a through-hole penetrating the insertion portion along the longitudinal direction thereof; and the sensor may be disposed on a side surface of the insertion portion.

By doing so, when the ligation loop is looped around the atrial appendage to perform ligation at a desired position, the distal end of the insertion portion is secured to the atrial appendage by the ligation loop. Therefore, the sensor, which is disposed on the side surface of the insertion portion, can also be secured to the atrial appendage, thus making it possible to confirm, with the sensor, the state of the atrial appendage before the ligation loop is cut.

Furthermore, in the above-described aspect, the distal end of the insertion portion may be provided with a groove that extends from an opening of the through-hole to a radially outer side and that can accommodate the ligation loop; and the sensor may be disposed on an outer surface of the insertion portion to which the groove is connected.

The ligation loop is exposed from the opening at the distal end of the insertion portion via the through-hole therein and is spread in any of the radial directions of the insertion portion along a plane intersecting the long axis of the insertion portion, thereby making it easy to be looped around the atrial appendage. In this case, the base end of the ligation loop exposed from the opening of the through-hole is accommodated in the groove, thereby making it possible to secure the phase of the insertion portion in the circumferential direction of the long axis of the insertion portion, with respect to the direction in which the ligation loop is spread, and to reliably make the sensor, which is provided on the outer surface of the insertion portion, face toward the atrial appendage.

Furthermore, in the above-described aspect, the insertion portion may have a flattened shape in cross-section; and the sensor may be disposed on a side surface that has a wider width.

By doing so, when the securing part, which is formed of the ligation loop looped around the atrial appendage, is tightened, thus securing the insertion portion to the atrial appendage, a wider surface of the insertion portion, which has a flattened shape in cross-section, is made to stably face the surface of the atrial appendage. Therefore, the sensor, which is provided on the wider side surface, can also be made to stably face the surface of the atrial appendage.

Furthermore, in the above-described aspect, an expansion part may be provided on an outer surface of the insertion portion; and the sensor may be disposed on an opposite side from the expansion part.

By doing so, the expansion part is activated, thereby making it possible to maintain a state in which the sensor, which is provided on the insertion portion, is stably brought into contact with the atrial appendage.

Furthermore, according to another aspect, the present invention provides an atrial-appendage ligation system including: an electrocardiograph that detects an electrocardiographic signal; one of the above-described atrial-appendage ligation treatment tools; and a determining unit that determines the state of ligation of an atrial appendage on the basis of the electrocardiographic signal detected by the electrocardiograph and a blood flow in the atrial appendage detected by the blood-flow sensor.

According to this aspect, it is possible to confirm whether a blood flow in the atrial appendage detected by the blood-flow sensor is right or wrong, by using an electrocardiographic signal detected by the electrocardiograph. Therefore, the ligation state of the atrial appendage can be determined more reliably by the determining unit.

Furthermore, in the above-described aspect, the determining unit may determine completion of the ligation when the blood flow to the atrial appendage does not exist during the systole in the electrocardiographic signal.

By doing so, even if a blood flow in the atrial appendage is detected by the blood-flow sensor, when the blood flow exists at a timing other than the systole in the electrocardiographic signal detected by the electrocardiograph, the detected blood flow can be determined to be noise. Therefore, ligation is performed until the blood flow does not exist during the systole, the ligation of the atrial appendage can be more reliably performed.

Furthermore, in the above-described aspect, the determining unit can determine that, when a heart rate in the electrocardiographic signal does not match a detected rate detected by the blood-flow sensor, either of the detected rates includes noise.

### REFERENCE SIGNS LIST

A atrial appendage
1 atrial-appendage ligation treatment tool
2 shaft (insertion portion)
2a through-hole
3 pressing portion (securing part)
4 contact part
5 sheath
6 blood-flow sensor (sensor)
8 ligation loop (securing part)
14 groove
16 electrocardiograph
17 determining unit
18 atrial-appendage ligation system

1. An atrial-appendage ligation treatment tool comprising:
an insertion portion that is introduced into a cardiac sac through a sheath penetrated through a pericardium;
a securing part that secures a distal end of the insertion portion to an atrial appendage; and
a sensor that is provided at the distal end of the insertion portion and that detects the state of the atrial appendage.

2. An atrial-appendage ligation treatment tool according to claim 1, wherein the sensor is a blood-flow sensor that detects a blood flow in the atrial appendage.

3. An atrial-appendage ligation treatment tool according to claim 2, wherein the blood-flow sensor is an ultrasound sensor.

4. An atrial-appendage ligation treatment tool according to claim 2, wherein the blood-flow sensor is an optical sensor.

5. An atrial-appendage ligation treatment tool according to claim 1, wherein the sensor is a hardness sensor that measures the hardness of atrial-appendage tissue.

6. An atrial-appendage ligation treatment tool according to claim 1, wherein the sensor is a color-measuring sensor that measures the color of atrial-appendage tissue.

7. An atrial-appendage ligation treatment tool according to claim 1, wherein the securing part comprises two contact parts that can be disposed at a position where the atrial appendage is to be sandwiched therebetween in the thickness direction.

8. An atrial-appendage ligation treatment tool according to claim 7, wherein the contact parts are provided such that the space therebetween can be changed.

9. An atrial-appendage ligation treatment tool according to claim 7, wherein the sensor is disposed at at least one of positions of the two contact parts that face each other.

10. An atrial-appendage ligation treatment tool according to claim 7, wherein the sensor is disposed at at least one of distal ends of the two contact parts.

11. An atrial-appendage ligation treatment tool according to claim 1,
wherein the securing part is a ligation loop that is introduced to the distal end of the insertion portion through a through-hole penetrating the insertion portion along the longitudinal direction thereof; and
the sensor is disposed on a side surface of the insertion portion.

12. An atrial-appendage ligation treatment tool according to claim 11,
wherein the distal end of the insertion portion is provided with a groove that extends from an opening of the through-hole to a radially outer side and that can accommodate the ligation loop; and
the sensor is disposed on an outer surface of the insertion portion to which the groove is connected.

13. An atrial-appendage ligation treatment tool according to claim 11,
wherein the insertion portion has a flattened shape in cross-section; and
the sensor is disposed on a side surface that has a wider width.

14. An atrial-appendage ligation treatment tool according to claim 11,
wherein an expansion part is provided on an outer surface of the insertion portion; and
the sensor is disposed on an opposite side from the expansion part.

15. An atrial-appendage ligation system comprising:
an electrocardiograph that detects an electrocardiographic signal;
an atrial-appendage ligation treatment tool according to claim 2; and
a determining unit that determines the state of ligation of an atrial appendage on the basis of the electrocardiographic signal detected by the electrocardiograph and a blood flow in the atrial appendage detected by the blood-flow sensor.

16. An atrial-appendage ligation system according to claim 15, wherein the determining unit determines completion of the ligation when the blood flow to the atrial appendage does not exist during the systole in the electrocardiographic signal.

17. An atrial-appendage ligation system according to claim 15, wherein the determining unit determines that, when a heart rate in the electrocardiographic signal does not match a detected rate detected by the blood-flow sensor, either of the detected rates includes noise.
